(19) United States
(12) Patent Application Publication
(10) Pub. No.: US 2003/0153029 A1
(43) Pub. Date: Aug. 14, 2003

(54) Bioactive substance secreted by canine transmissible venereal tumor with mammalian B lymphocyte toxicity

(76) Inventors: Rea-Min Chu, Taipei (TW); Kuang-Wen Liao, Taipei (TW); Shao-Wen Hung, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/072,922

(22) Filed: Feb. 12, 2002

(51) Int. Cl. C12P 1/00; A61K 35/12

(52) U.S. Cl. 435/41; 424/520

(57) ABSTRACT

The present invention herein relates to a bioactive substance with mammalian B lymphocyte toxicity secreted by canine transmissible venereal tumor (CTVT) and its process. CTVT is the only mammalian tumor in nature that is transmitted through viable tumor cells. Transmission of CTVT between dogs is akin to an allograft. The isolation of bioactive substance secreted by CTVT includes the following steps: (1) store CTVT excised from canine skin in Hank's balanced salt solution (HBSS); (2) physically extract CTVT, tumor infiltrating lymphocytes and peripheral blood lymphocytes; (3) place CTVT in culture medium and physically obtain suspension fluid with B lymphocyte toxicity; (4) drive said suspension solution through protein filters with different pore sizes to obtain bioactive substance. Said bioactive substance secreted by CTVT and having specific toxicity for mammalian B lymphocytes is a protein substance with a molecular weight between 30˜100 kD and free of α-Dmannosyl and α-D-glucosyl residues and it is soluble, heat-sensitive and proteinase K-sensitive.

### BACKGROUND OF THE INVENTION

1) Field of the Invention

The invention herein relates to a bioactive substance with mammalian B lymphocyte toxicity secreted by canine transmissible venereal tumor (CTVT) and its process, said bioactive substance has specific toxicity for mammalian blood B lymphocytes.

2) Description of the Prior Art

Illnesses caused by hyperactivity of B lymphocytes include asthma, allergy and all kinds of auto-immune diseases. Neoplasm in lymphatic tissues is also characterized by the destruction of normal structure of lymph nodes where morphologically mature or immature lymphocytes proliferate continuously and the cancerous cells can metastasize to other tissues along the lymph system. Malignant lymphatic tumors are commonly referred to as lymphoma. The incidence of lymphoma is similar in different age groups and gender groups. The management of lymphoma includes surgical excision and radiation, but relapse and metastasis are common. Early-stage lymphoma may be treated by radiation using Cobalt 60, while late-stage, aggressive and relapsed lymphoma are often managed by radiation and chemotherapy. These approaches adversely affect normal body tissues while killing cancerous cells. The recently developed bone marrow or peripheral blood stem cell transplantation for the treatment of lymphoma also has its limitations, such as the difficulty of finding donor and the problem of host rejection, matching and histocompatibility. Thus how to regulate the activity of B lymphocytes and come up with effective therapeutic measures to benefit patients of malignant diseases should be the focus direction for researchers and parties concerned.

In view of the problems just discussed, the author proposes here a novel process for bioactive substance with mammalian B lymphocyte toxicity secreted by canine transmissible venereal tumor based on many years of research, development, professional knowledge and sales experience in related products.

### SUMMARY OF THE INVENTION

The primary objective of the invention herein is to provide a process for making bioactive substance secreted by canine transmissible venereal tumor (CTVT) which has specific toxicity to mammalian B lymphocytes.

Another purpose of the invention is to provide a bioactive substance which is secreted by canine transmissible venereal tumor and has specific toxicity to mammalian B lymphocytes.

The lymphatic system consists of mainly B lymphocytes and T lymphocytes. Dogs inoculated with CTVT will see a substantial reduction of B lymphocytes in peripheral blood. But after all tumors are excised, the levels of B lymphocytes and immunoglobulins are back to normal. Furthermore, it is found that CTVT secrets a soluble, heat-sensitive and proteinase K-sensitive substance with B lymphocyte-specific toxicity, which causes the proportional drop of blood B lymphocytes. Moreover, CTVT also exhibits inhibitory action against the B lymphocytes of humans, cats, horses and mice. Thus its secretion may be applied in the regulation of B cells for treatment of related diseases.

Detailed descriptions of a preferred embodiment are provided below to depict the theory and technical features of this invention for reference of people familiar with the technique. Nevertheless the claims of this invention are not restricted by the presentation of preferred embodiments below.

### DETAILED DESCRIPTION OF THE PREFERRED EMBODIMENTS

CTVT is the only type of tumor in nature that is transmitted through viable tumor cells. It may be classified as a type of allograft by its growth characteristics. CTVT is a naturally occurring tumor seen in dogs and all over the world, particularly in areas where roaming dogs are abound. It may be transmitted through cell transplantation during coitus, or the contact of mucosa or blood in the course of scratching, licking, or biting. CTVT often occurs in external genitalia of adult dogs. It is mostly seen in the penis and the glans penis of male dogs and the anterior part of the vagina, cervix, uterus, and uterine tube of female dogs. Extragenital occurrence of CTVT has been reported in skin and oral mucosa.

1. In-Vivo Study of Tumor Growth:

Six dogs, each received 12 inoculations of 1×10⁸ freshly-isolated CTVT cells subcutaneously on the back. Tumor size was measured once every week. Blood tests were performed once every two weeks and CTVT tissues were surgically removed for histological examination once every two to three weeks. All samples were stored in Hank's balanced salt solution (HBSS).

Take 10 g of CTVT tissue, mince it in 90 ml of HBSS solution by crushing it with No. 25 stainless steel net and obtain a monocellular suspension after filtration. Layer 8 ml of suspension in 4 ml of 42% Percoll™ gradient solution and undergo centrifugation for 25 minutes under 18° C. and 800 g; wash the resulting solids three times with MEM solution (Gibico) to obtain CTVT and tumor infiltrating lymphocytes (referred to as TIL below).

To separate peripheral blood lymphocytes (called PBL below), collect peripheral blood samples before CTVT inoculation, once every two weeks after CTVT inoculation and one year after complete removal of CTVT and obtain PBL by Ficoll gradient method as follows: layer 4 ml of blood sample in 3 ml Ficoll gradient solution and subject it to centrifugation for 20 minutes under 18° C. and 800 g; wash the resulting solids with fluorescent antibody buffer (FAB) three times to obtain PBL.

2. Flow Cytometer Analysis

Incubate the cells in anti-CD21 (marker) specific monoclonal antibody and subject them to indirect immunofluorecence staining. Measure fluorescence intensities of the viable cells with flow cytometer.

3. In-Vitro Study of B Cell Toxicity

Take 2×10⁶ /ml CTVT cells and culture them in Dubecoo's modified Eagle medium (DMEM) containing 10% fetal calf serum (FCS) under 37° C. and 5% CO₂ for three days; (1) take out top-layer suspension fluid or subject it to boiling water bath for 5 minutes, and then have both separately interact with the PBL of normal dogs to observe the cytotoxicity of said fluid; (2) add 10 μg of proteinase K to freshly cultured top layer suspension fluid under 37° C. overnight and let it interact with the PBL to observe the cytotoxicity of said fluid to B cells.

4. The Effect of Substance Secreted by CTVT on other Mammalian B Lymphocytes

Culture the aforesaid top-layer suspension fluid with the B cells of humans, house cats, houses or BALB/c mice and observe the effect of bioactive substance in CTVT on non-canine mammalian B lymphocytes by flow cytometry.

5. Characteristics of CTVT-Secreted Substance

(1) Filter cultured medium of CTVT cells that have been cultured for 72 hours using Centriplus tubes with different pore sizes (for molecular weight cut-offs of 4, 10, 30, and 100 kD); (2) take 200 μl top-layer suspension that passed through 100 kD molecular weight cut-off, add 400 μl Con A Sepharose to culture for one hour, and then remove the gel by centrifugation to analyze the molecular weights of substances in the suspension and their reactions to Con A Sepharose.

6. Statistical Analysis

Data are analyzed using Student's t-test. Results with p values less than 0.05 are considered to be significantly different.

FIG. 1 illustrates the effect of CTVT on the level of peripheral blood B lymphocytes (PBBL) in dogs as described in the preferred embodiment above before and after CTVT inoculation. As shown, CTVT sustained 4˜6 months of fast growth in host body which is characterized by a progression phase (called P phase below), followed by a regression growth (called R phase below). Throughout the growth phase of CTVT, it is observed from the blood of the inoculated dogs that PBBL sustained linear decline where the B cell levels in P phase and R phase after inoculation were markedly lower than that before inoculation (Nb) (p<0.05 is indicated by the symbol of *) and B cell level in R phase was more markedly lower than that in P phase ((p<0.05 is indicated by the symbol of #). But the proportion of B cells in blood went back to normal after complete excision of tumors (Na).

FIG. 2 depicts the effect of CTVT on the level of B cells in TIL in dogs as described in the preferred embodiment above before and after CTVT inoculation. As shown, the P phase and R phase of CTVT growth could be further divided into two phases respectively—Pb (3˜8.5 weeks after inoculation) and Pa (10.5˜19 weeks after inoculation), Rb (19˜22 weeks after inoculation) and Ra (22˜25 weeks after inoculation). After CTVT inoculation, B cell level in TIL exhibited linear decline from Pb, Pb, Rb to Rb phase. It could be deduced that CTVT during its growth phase after inoculation must have escaped the attack of the host immune system through some unknown mechanisms.

FIG. 3A depicts the comparison of the concentration of serum immunoglobulin M (IgM) in dogs described in the preferred embodiment before and after CTVT inoculation. As shown, the density of serum IgM exhibited linear decline from pre-inoculation to P phase and R phase after inoculation; the IgM density in P phase and R phase showed significant difference respectively as compared to that before inoculation (N) (p<0.05 is indicated by the symbol of *), and the IgM densities in R phase and P phase exhibited significant difference as compared with each other (p<0.05 is indicated by the symbol of #). FIG. 3B illustrates the comparison of the concentration of serum immunoglobulin G (IgG) in dogs described in the preferred embodiment above before and after CTVT inoculation. As shown, the density of IgG also displayed linear decline and those of R phase and P phase showed significant difference (p<0.05 is indicated by the symbol of#).

FIG. 4A depicts the effect of suspension fluid obtained from CTVT culture on B cell level before and after said suspension fluid has been treated with boiling water bath as described in the preferred embodiment above. As shown, after the in-vitro cultured CTVT suspension fluid was treated with boiling water bath and added into the peripheral blood, the level of peripheral B cells is comparable to that of the non-inoculated control group (MEM) both in P phase (CTVT-P Boil) and R phase (CTVT-R Boil), indicating that the bioactive substance secreted by CTVT would lose its activity under heat. Referring to FIG. 4B which depicts the addition of proteinase K to cultured CTVT suspension fluid on the level of B cells, it is found that after the CTVT was added with proteinase K, the level of peripheral B cells is comparable to that of the non-inoculated control group (MEM) both in P phase (CTVT-P Prot K) and R phase (CTVT-R Prot K), and in the control group that was added with proteinase K, the level of peripheral B cells is also comparable to that of the non-inoculated control group (MEM), indicating that proteinase K which breaks down protein molecules would cause the bioactive substance in CTVT to lose B cell toxicity, meaning said bioactive substance is either a protein or a substance with protein functions.

Referring to FIG. 5A which compares the death rate of B cells caused by the CTVT-secreted bioactive substance before and after boiling as described in the preferred embodiment above, it is found that after boiling which caused the denaturation of the CTVT-secreted bioactive substance, the bioactive substance in CTVT showed comparable toxicity to B cells after boiling as compared to that of the control group (MEM) in either P phase or R phase (P Boil or R Boil). Comparatively, CTVT-secreted bioactive substance that was not subject to boiling exhibited far greater B cell toxicity in either P phase or R phase as compared to that of the control group (MEM) or the denatured bioactive substance after boiling in either P phase or R phase (P-Boil or R-Boil).

Referring to FIG. 5B which also compares the B cell specific death rate caused by the CTVT-secreted bioactive substance before and after boiling as described in the preferred embodiment above, the cross axis represents the content of CD21 marker and the vertical axis represents death rate (PI). It is seen in the upper right quadrant of FIGS. a, b, c and d of FIG. 5B that the bioactive substance only causes high death rate of B cells but not that of non-B cells, suggesting that the bioactive substance secreted by CTVT had B cell-specific toxicity.

FIG. 6 illustrates the effect of CTVT-secreted bioactive substance on the level of peripheral B cells of humans, house cats, horses or mice. It is observed that CTVT secretion in either P phase or R phase would cause a decrease of peripheral B cell level in humans, house cats, horses or BALB/c mice. But after being subjected to boiling water bath, said bioactive secretion exhibited no inhibition against the peripheral B cells of humans, house cats, horses or mice, indicating that said bioactive substance has peripheral B cell-specific toxicity in mammals of different species.

If subjecting the cultured CTVT suspension fluid to Centriplus filters of different pore sizes (molecular weight cut-offs of 4, 10, 30 and 100 kD), it is found that said suspension fluid with molecular weight of 30˜100 kD still showed B cell toxicity, while that with molecular weight less than 30 kD or greater than 100 kD did not have B cell toxicity, suggesting that the bioactive substance with B cell toxicity had a molecular weight between 30˜100 kD. If culturing said suspension fluid having molecular weight between 30˜100 kD with 400 μl Con A Sepharose which can remove α-D-mannosyl and α-D-glucosyl, it is found that the B cell toxic bioactive substance in said suspension fluid is a glycosyl-free protein.

To further analyze said 30˜100 kD protein using high performance liquid chromatography (HPLC), the results are shown in FIG. 7A. The solution in each test tube was then tested for B cell toxicity, and the one showing the greatest toxicity was subjected to SDS-gel electrophoresis (tube No. 30 with a * mark as shown in FIG. 7B) and molecular weights obtained were 68, 77.1, 86.5 and 91.4 kD respectively (as shown in FIG. 8).

The experiments described above show that the bioactive substance secreted by CTVT has specific toxicity to mammalian B lymphocytes and may be used in the regulation of B cells and the treatment of B cell related disorders, such as allergy (asthma) and B cell lymphoma.

In summary, the invention herein relates to a bioactive substance secreted by canine transmissible venereal tumor that has toxicity to mammalian B lymphocytes and its process, particularly a bioactive substance secreted by canine transmissible venereal tumor having specific toxicity to mammalian B lymphocytes. This invention is novel, progressive and may be used by the industry, and should be meeting the criteria for patent application.

What has described above is merely one preferred embodiment of the invention herein, which should not be construed as a limitation on the actual applicable scope of the invention herein and as such, all modifications based on adaptations of the shape, structure, features and spirit contained in the above detailed descriptions and claims below shall remain within the protected scope and claims of the invention herein.

### BRIEF DESCRIPTION OF THE DRAWINGS

FIG. 1 is a chart comparing the effect of CTVT on the level of B cells in peripheral blood of dogs as described in a preferred embodiment of the invention herein before and after CTVT inoculation.

FIG. 2 is a chart comparing the effect of CTVT on the level of B cells in TIL in dogs as described in a preferred embodiment of the invention herein before and after CTVT inoculation.

FIG. 3A is a chart comparing the concentration of serum immunoglobulin M in dogs as described in a preferred embodiment of the invention herein before and after CTVT inoculation.

FIG. 3B is a chart comparing the concentration of serum immunoglobulin G in dogs as described in a preferred embodiment of the invention herein before and after CTVT inoculation.

FIG. 4A is a chart comparing the effect of CTVT suspension fluid on the level of B lymphocytes before and after boiling water bath as described in a preferred embodiment of the invention herein.

FIG. 4B is a chart comparing the effect of CTVT suspension fluid on the level of B lymphocytes before and after the addition of proteinase K as described in a preferred embodiment of the invention herein.

FIG. 5A is a chart comparing the effect of bioactive substance secreted by CTVT on the death rate of B lymphocytes before and after the treatment of boiling water bath as described in a preferred embodiment of the invention herein.

FIG. 5B is a chart comparing the effect of CTVT-secreted bioactive substance of a preferred embodiment of the invention herein on the specific cytotoxic ability of aforesaid substance B lymphocytes before and after the treatment of boiling water bath.

FIG. 6 is a chart comparing the effect of CTVT-secreted bioactive substance of a preferred embodiment of the invention herein on the level of peripheral blood B lymphocytes of humans, house cats, horses and mice.

FIG. 7A is a chromatogram showing the analysis result of CTVT-secreted bioactive substance of a preferred embodiment of the invention herein using HPLC.

FIG. 7B is a chart comparing the effect of CTVT-secreted bioactive substance of a preferred embodiment of the invention herein on canine peripheral blood B lymphocytes.

FIG. 8 is the SDS-gel electrophoresis result of the solution in FIG. 7B that showed the greatest toxicity to canine peripheral blood B lymphocytes.

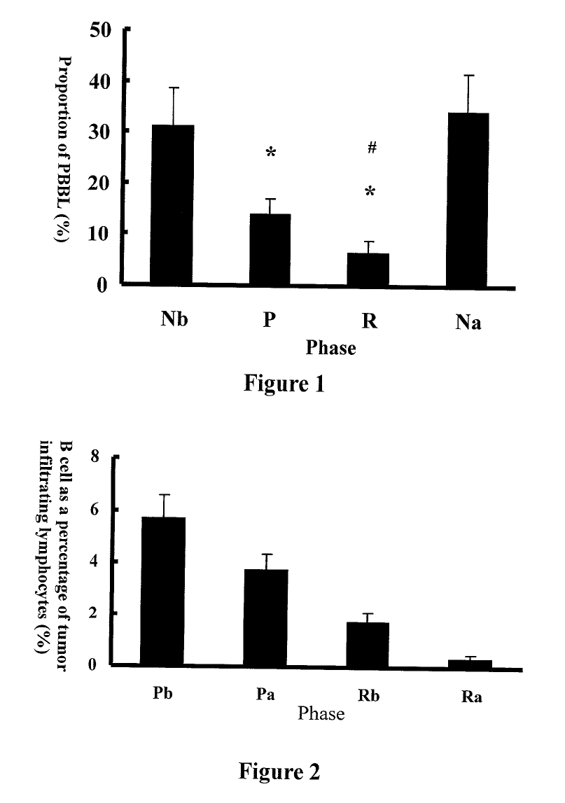

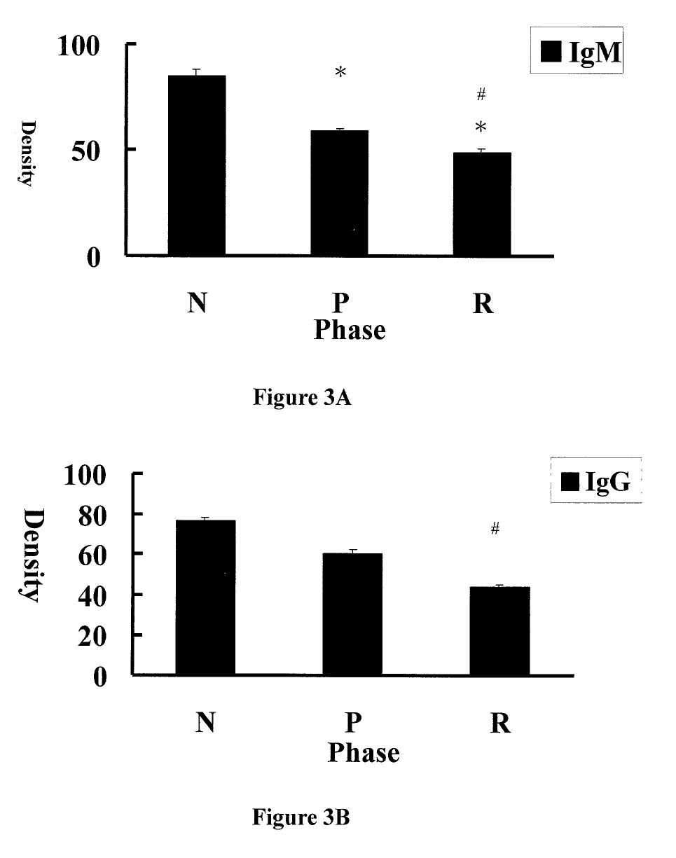

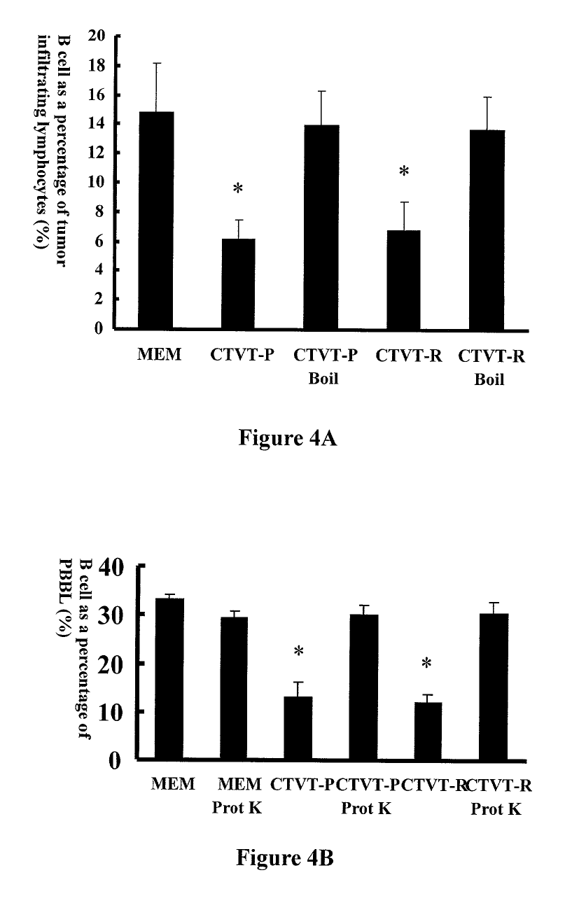

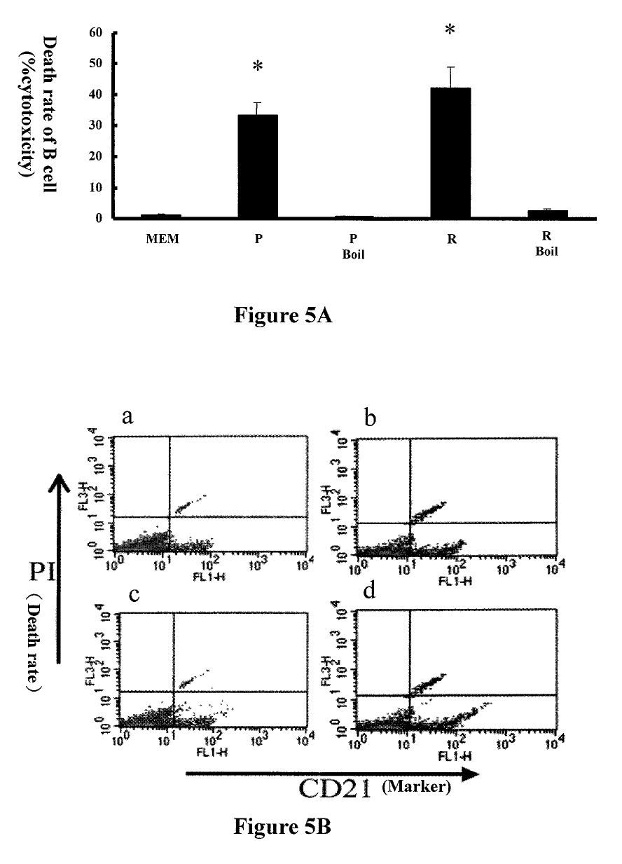

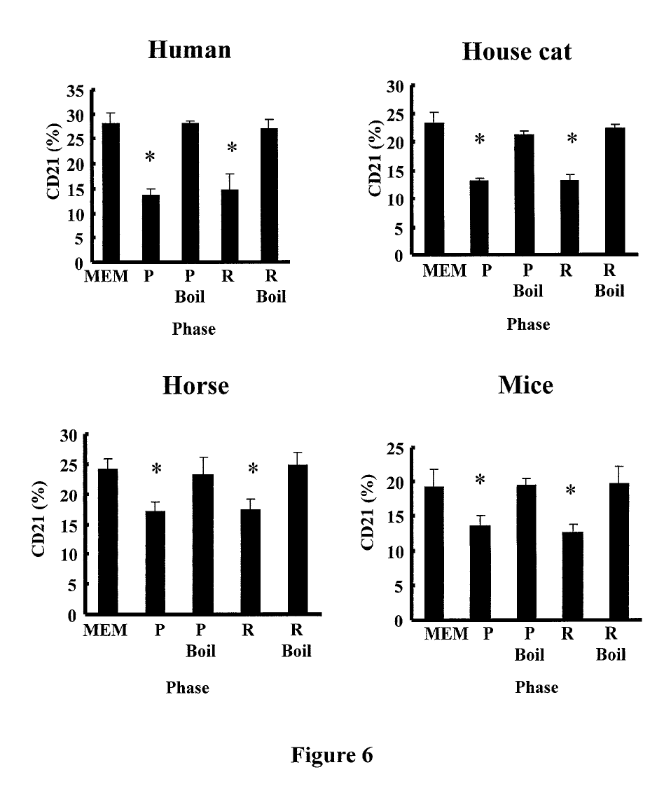

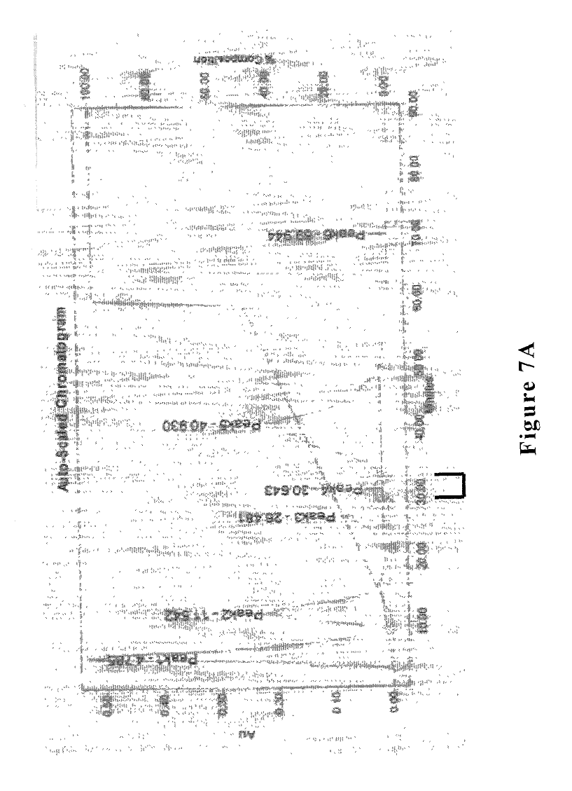

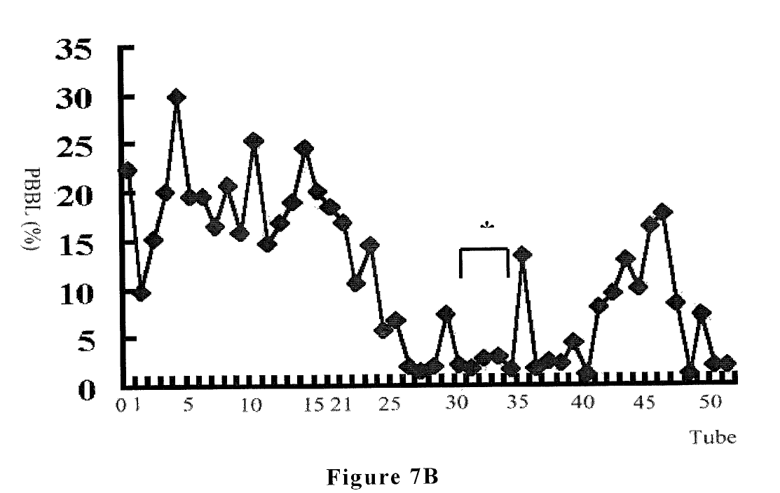

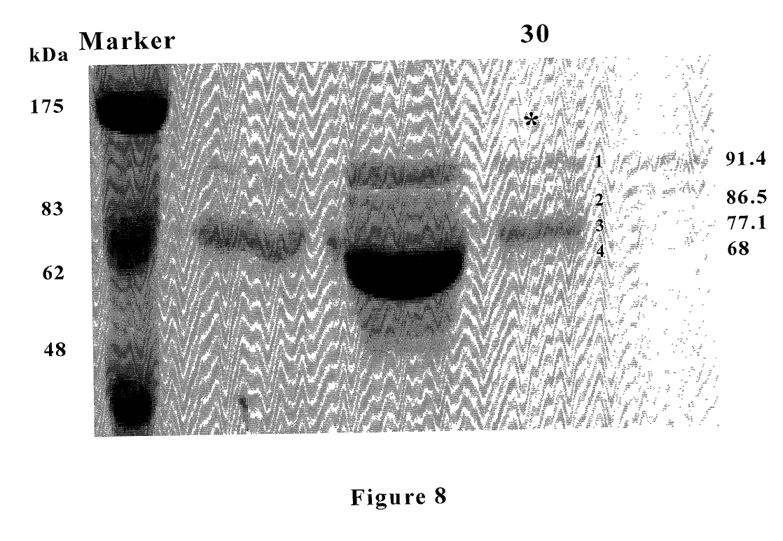

1. A process for bioactive substance secreted by canine transmissible venereal tumor with mammalian B lymphocyte toxicity, featuring the steps of:
(1) storing canine transmissible venereal tumor (referred to as CTVT below) excised from the skin of affected dogs in HBSS (Hank's balanced salt solution);
(2) screening and obtaining CTVT, tumor infiltrating lymphocytes and peripheral blood lymphocytes using physical methods;
(3) putting CTVT in culture medium and obtaining suspension fluid with B lymphocyte toxicity using physical methods; and
(4) driving said suspension fluid with B lymphocyte toxicity through protein filters of different pore sizes.

2. A process for bioactive substance as mentioned in claim 1, in which the culture in step (3) is carried out under an environment 35˜39° C.

3. A process for bioactive substance as mentioned in claim 2, in which the preferred temperature of culture in step (3) is 37° C.

4. A process for bioactive substance as mentioned in claim 1, in which the culture is step (3) is carried out under an environment of 4˜6% of CO₂.

5. A process for bioactive substance as mentioned in claim 4, in which the preferred culture condition in step (3) is 5% CO₂.

6. A process for bioactive substance as mentioned in claim 1, in which the culture in step (3) is carried out in vitro for 1˜5 days.

7. A process for bioactive substance as mentioned in claim 6, in which the preferred culture condition in step (3) is in vitro for 3 days.

8. A kind of bioactive substance secreted by CTVT having mammalian B lymphocyte toxicity, in which said bioactive substance having specific toxicity to mammalian B lymphocytes is a protein with molecular weight of 30˜100 kD and free of α-D-mannosyl and α-D-glucosyl residues and soluble, heat-sensitive and proteinase K-sensitive.

9. A kind of bioactive substance as mentioned in claim 8 in which the molecular weight of said substance lies between 65˜95 kD.
